(19) United States
(12) Patent Application Publication
(10) Pub. No.: US 2017/0219551 A1
(43) Pub. Date: Aug. 3, 2017

(54) MOUNT APPARATUS FOR A SUBMERSIBLE ANALYZER AND METHOD FOR ANALYZING FLUID

(71) Applicant: D.C. Water & Sewer Authority, Washington, DC (US)

(72) Inventors: Salil Kharkar, Washington, DC (US); Nicholas Passarelli, Washington, DC (US); Chris Reilly, Washington, DC (US); Michael Nye, Washington, DC (US); Michael Chen, Washington, DC (US); James L. Clarke, Washington, DC (US); Biju George, Washington, DC (US); Sudhir N. Murthy, Herndon, VA (US)

(21) Appl. No.: 15/422,225

(22) Filed: Feb. 1, 2017

Related U.S. Application Data
(60) Provisional application No. 62/289,502, filed on Feb. 1, 2016.

(51) Int. Cl. G01N 33/18; G01D 11/24; F16M 11/22

(52) U.S. Cl. CPC G01N 33/1886; F16M 11/22; G01D 11/245

(57) ABSTRACT

A mount or submersible or semisubmersible housing for supporting a submersible analyzer or device and method for analyzing fluid. The mount includes an elongated submersible housing that supports the analyzer or device. The housing is ruggedized and has a geometric body with an internal cavity and with upper and lower ends. The upper end is configured to mount to a fixed structure. In some embodiments a slot extends between the upper and lower ends of the housing along a longitudinal axis thereof. In some such embodiments the slot is sized to receive a portion of a sliding extension that supports a sensor of the analyzer or device, thereby facilitating the installation and removal of the analyzer or device with respect to the elongated submersible housing. An attachment may be used to fixedly mount the elongated submersible housing to the fixed structure.

### RELATED APPLICATION

This application claims priority to U.S. provisional application No. 62/289,502 entitled Mounting and Maintaining Wastewater Analyzers, filed on Feb. 1, 2016, the entire disclosure of the provisional application is incorporated herein by reference.

### FIELD OF THE INVENTION

The present invention relates to the design, installation, and use of multifunctional stabilizing mounts, or housings which may be attached to mounts, for use in special cases as a support for submersible or semi-submersible analyzers (sensors and probes and their inputs and outputs), including other devices that may need to be periodically removed from the environment such as a velocity meter or micro hydraulic turbine, broadly in any fluid environment (gas or liquid), and particularly in environments where the anticipated velocity of fluids is substantial and/or the mounting depth of the devices is significant. Such analyzers and devices may include but are not limited to sensors or probes for taking physical measurements including but not limited to flow and velocity, or for energy generation including but not limited to micro hydraulic turbines or solar panels, or for measuring, for example, the flow of ammonia, nitrates or nitrites in environments, including but not limited to water, wastewater, rivers, lakes, reservoirs, distribution and collection systems, chemical plants, or power plants, hospitals and hotels; and in air (for example, nitrogen oxides, sulfur oxides and particulates) for cities, towns, industries, factories and in the indoor air environment. The sensing can be visual, physical, or spectral (such as a camera or any spectral imaging such as ultra-violet, infra-red, Raman or FTIR), auditory (sound), vibratory, potentiometric, or in the chemical environment (analysis of chemical species). These housings or mounts also support the ruggedization of existing mounts, sensors, analyzers or other multifunctional device including those associated with computation, connectivity, communication protocols, infrastructure protocols, identification protocols, communications/transport protocols (ex: Wi-Fi, Bluetooth, LPWAN), discovery protocols, data protocols, device management protocols, semantic protocols, multi-layer framework protocol, security protocol, process automation protocols, protocol translators, protocol convertors, energy generators, and energy storage.

### BACKGROUND OF THE INVENTION

The water/wastewater industry has been faced with problems involving sub-standard mounts for in-situ analyzers and devices. This is particularly true for submersible analyzers and devices that need to be deep, e.g. that hang 10 or more feet below the surface of the platform or working deck. This is also known as long drops. The mounts also currently provide limited functionality and are less rugged.

U.S. Pat. No. 7,942,031 to Tranquille discloses a shock resistant mounting for analyzers, such as carbon monoxide detectors and fire detectors which do not require long drops. Thus, the mounting of Tranquille would be ill suited for uses involving analyzer stabilization at a distance and high fluid velocity. Other mounting solutions have involved brackets located above the analyzers. The problem with these brackets is that they are too far from the analyzer to provide stability, are often flimsy, and break easily in high velocity flows. U.S. Pub Appln No. 2014/0090467 to Carter discloses a specialized fixed mounting device for supporting sensors that can detect parameters with a machine or a rotating member, but does not address use with fluids or its flow. U.S. Publ. Appln No. 2015/0107379 to Rose et al. describes a mount for an environmental sensor that has limited applicability associated with stabilization at a distance. The current state of mounts and associated attachments offer limited functionality and applicability for fluid flow.

Another problem with analyzers today is that they are typically bundled instruments with a sensor, a transmitting protocol, a monitoring device, and a controller. The bundling of these instruments increases costs for the end user with limited additional value.

U.S. Publ. Appln. No. 2005/0031490 to Gumbrecht et al. discloses an assemblage or module for biochemical analysis consisting of a sensor chip, electrical contacts and fluid for flow between the chip and contacts. This assemblage provides for decentralized biochemical analysis, but does not support any other functional attributes that provides for mechanical accessorization with multifunctional attributes.

As such, a mounting device or accessory that assists in the translation and cleaning of signal, and/or conversion of this signal into a uniform protocol, aggregates information from multiple sensors and/or transmission of this information is needed. This translation and transmission will facilitate the use of iOT concepts, edge computing, distributed control, or improved central management of data. It will also reduce the overall price of a sensor and control systems. Also, mount or mount accessory that can stabilize a sensor possibly subject to high velocity or deep sensing, and/or at the same time provided multifunctional ‘smart sensing’ attributes as needed in the marketplace.

Therefore, a need also exists for a multifunctional mount or a submersible housing for sensors analyzing fluids. Submersible analyzers that maintain the stability and protection of the analyzer in high velocity fluids is also needed. The need also exists for such a mounting or a submersible housing that facilitates the removal of a sensor or analyzer to the working deck for the purposes of performing periodic analyzer maintenance. Finally, there exists a need for the mount or submersible housing to provide additional functionality by providing part or all of the associated computation and/or connectivity.

### SUMMARY OF THE INVENTION

Accordingly, the present invention provides a multifunctional mount or a submersible housing for supporting a fluid sensor/analyzer, in certain embodiments being a submersible analyzer that comprises an elongated submersible or semi-submersible housing for supporting an analyzer or device. The elongated submersible housing is ruggedized. Ruggedized means the housing is formed of a rigid support material that is also corrosion and weathering resistant and waterproof, and supports the analyzer or sensor to prevent breakage or damage thereof or prevent the analyzer or sensor from being destroyed by the fluid in which it is submerged. The housing has a geometric body that in certain embodiments has an internal cavity to slide the analyzer mount with upper and lower ends. The upper end is configured to mount to a fixed structure, such as a working deck or any permanent or temporary extension (including but not limited to a handrail or tripod) from a fixed surface, and is open to receive the analyzer into the internal cavity in these embodiments. In some such embodiments, a slot extends between the upper and lower ends of the elongated submersible housing along a longitudinal axis thereof where the slot is sized to accept a mount that supports a sensor of the analyzer or another device. In one embodiment, an attachment is provided that fixedly mounts the housing to the fixed structure. The attachment may be a bonded engagement, for example, between an outer surface of the housing and a base plate on the fixed structure. In another embodiment, the upper end of the housing has an opening for installing the analyzer or device and the lower end is at least partially closed. In an alternative embodiment, the lower end may be substantially closed. In yet another embodiment, at least one spacer is disposed around the extension, where the spacer has at least one radial arm that extends between the extension and the inner surface of the housing. In a preferred embodiment, at least three spacers are disposed around the extension and the spacers are spaced from one another and each spacer has at least one radial arm that extends between the extension and the inner surface of the housing.

In a preferred embodiment, the housing is formed of a rigid material, such as stainless steel, but other embodiments do not restrict it to a specific material of construction. The multifunctional capability provides for the management of associated computation, connectivity, communication protocols, infrastructure protocols, identification protocols, communications/transport protocols (ex: Wi-Fi, Bluetooth, LPWAN), discovery protocols, data protocols, device management protocols, semantic protocols, multi-layer framework protocol, process automation protocols, protocol translators, security protocol, protocol convertors, energy generators, energy storage. This multifunctional mount or submersible housing facilitates the hosting of a branded or generic sensor, analyzer or other devices.

The computation associated with the sensor or devices of the present invention can reside within the accessory in a chipset housed within a hardened case or industrial personal computer. The mount or submersible housing can also support a storage or energy conversion device such as a battery, a capacitor, a turbine, or any such means to facilitate the use of a sensor where a power connection is not easily available.

The preferred embodiment of the present invention also provides a mount for supporting a submersible analyzer and other devices that need to be supported and that comprises an elongated submersible housing that supports the analyzer and any number of devices. The housing is formed of a rigid material. The elongated submersible housing has a geometric body that has an internal cavity to slide the analyzer mount with upper and lower ends. The upper end is configured to mount to a fixed structure. A slot extends between the upper and lower ends of the elongated submersible housing along a longitudinal axis thereof. The slot is sized to receive a portion of an extension that supports a sensor of the analyzer or device, thereby facilitating the installation and removal of the analyzer or device with respect to the elongated submersible housing. An attachment fixedly mounts the elongated submersible housing to the fixed structure. In a preferred embodiment, the attachment is a weld between the housing and the fixed structure.

In certain embodiments, the present invention further provides a method of stabilizing an analyzer or device submersible from a fixed structure that comprises the steps of fixedly mounting an upper end of an elongated submersible housing to the fixed structure, the elongated submersible housing having a geometric body formed of a rigid material with a cavity running through it; with an internal sliding extension that supports a sensor of the analyzer or another device; installing the analyzer or device into an opening at the upper end of the elongated submersible housing so that a lower portion of the sliding extension passes through a slot extending between the upper end and a lower end of the elongated submersible housing along a longitudinal axis thereof; and advancing the analyzer or device down toward the lower end of the elongated submersible housing such that the sensor or device is exposed outside of the elongated submersible housing near or at the lower end thereof and the at least one spacer is located between the sliding extension and an inner surface of the elongated submersible housing.

The present invention also provides a method of analyzing fluid from a submersible or semi-submersible housing that comprises the steps of installing one or more sensors within the housing; installing one or more local computing devices or installing one or more relays or transmitters to transmit a signal from said one or more sensors to one or more remote computing devices or a cloud network, said local and remote computing devices communicating with said one or more sensors either through wired means or wireless means; and positioning the housing such that the one or more sensors is in fluid and transmits data regarding properties of the fluid to said one or more local or remote computing devices. The cloud or cloud network is a communications network using remote servers hosted in the Internet to store, manage, and process data.

The method of analyzing may also include the step of stabilizing the housing from a fixed structure by fixedly mounting an upper end of the housing to the fixed structure, the housing having a geometric body that is ruggedized; coupling at least one spacer with a sliding extension that supports a sensor of an analyzer or device; installing the analyzer or device into an opening at the upper end of the housing so that a lower portion of the extension passes through a slot extending between the upper end and a lower end of the housing along a longitudinal axis thereof; and advancing the analyzer or device down toward the lower end of the housing such that the sensor is exposed outside of the housing near or at the lower end thereof and the at least one spacer is located between the extension and an inner surface of the housing.

The method of analyzing may further include the steps of utilizing the one or more computing devices to implement complex protocols; and/or coupling an energy generation device to the housing for the generation of energy; and/or installing an energy storage device for the collection of energy; and/or sending a signal from the housing to the one or more remote computing devices, wherein the wireless means of communication is near field communication or an antenna; and/or providing one or more rigid port-slots in the housing for insertion and removal of one or more computing devices, energy storage devices or energy generation devices, wherein the one or more port-slots are integrated into the housing in order to facilitate the ruggedization of the sensors and the devices; wherein the port slots connecting the housing to the mount incorporates circuitry to facilitate the transmission of power or data from or to the housing.

The present invention yet further provides a submersible or semi-submersible housing that comprises a ruggedized housing body that supports one or more sensors, one or more relays capable of communicating with one or more computing devices, one or more port-slots for connecting the ruggedized housing body to a mount or one or more slots for the installation and removal of the one or more sensors wherein the slots are rigidly integrated into the housing to facilitate the ruggedization of the one or more sensors, and one or more computing devices capable of implementing complex protocols, or any number of energy storage devices, or any number of energy generation devices. The submersible or semi-submersible housing may also include an interface panel for direct control of any of the sensors therein, and any number of buttons for cycling between the various sensors, display options for the various sensors, or the sensors and display options of the sensors, wherein the one or more sensors are velocity sensors and the any number of energy generation devices are micro hydraulic turbines. In one embodiment, the one or more computing devices are supported by the housing body, wherein the one or more sensors sends a signal to the one or more computing devices directly through either wired or wireless means. In another embodiment, the one or more relays sends a signal from the one or more sensors to the one or more computing devices by wireless means. In certain embodiments, the housing body includes at least one of the any number of an energy generation devices, and/or at least one of the any number of energy storage devices. The housing body may include the one or more slots for insertion and removal of a plurality of computing devices, energy storage devices, or energy generation devices, wherein the one or more port-slots are integrated into the housing in order to facilitate the ruggedization of the one or more sensors.

In certain embodiments the port slots on the submersible housing may be connected to port slots on a mount linking the two together and attaching the housing to said mount for stabilization purposes. In some such embodiments the slots on the mount and/or submersible housing may include a means to connect power or data from or to the submersible housing, including but not limited to a quick connect, plug or outlet.

In certain embodiments the submersible housing may include a display panel which can be used to display information regarding the housed sensors, devices or analyzers. Data related to these sensors, devices or analyzers may be cycled using one or more buttons on the housing. In some such embodiments the display panel may be an interface panel which can be used to control any of the sensors, analyzers or devices for purposes including but not limited to a sensor taking an immediate reading, changing the type of information being sensed, taking extended readings, a device latching into place, locking into position, unlocking, sending collected energy or powering on, or an analyzer analyzing collected data, preparing to analyze data to be collected at a prescribed time, or changing the type of data being analyzed.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawing figures:

FIG. 1 is a front elevational view a submersible analyzer or device mount according to an exemplary embodiment of the present invention, showing the submersible analyzer or device mount being used with two different analyzers;

FIG. 2A is an enlarged partial front elevational of a housing of the submersible analyzer or device mount illustrated in FIG. 1, showing the housing without the analyzer;

FIG. 2B is a cross-sectional top view of the housing illustrated in FIG. 2A;

FIG. 3 is a top plan view of the housing illustrated in FIG. 2A, showing a spacer disposed in the housing and around the analyzer;

FIG. 4 is a bottom plan view of the housing illustrated in FIG. 2A;

FIG. 5 is an elevational view of an exemplary analyzer supported by the submersible analyzer mount illustrated in FIG. 1, showing the analyzer without the mount;

FIG. 6 is an elevational view of another exemplary analyzer supported by the submersible analyzer mount illustrated in FIG. 1, showing the analyzer without the mount;

FIG. 7 is an isometric view of a cross-section of the housing submerged in fluid, such as a liquid, showing the housing protecting sensors that are collecting data regarding the fluid and sends the data through a relay to a computing device wirelessly, while a turbine is used to generate energy; and

FIG. 8 is another isometric view of the housing submerged in a fluid, such as air, showing the housing protecting sensors that send a signal to an onboard computing device while energy is generated via a solar panel on the device and collected in an on board battery.

### DETAILED DESCRIPTION OF THE PREFERRED EMBODIMENTS

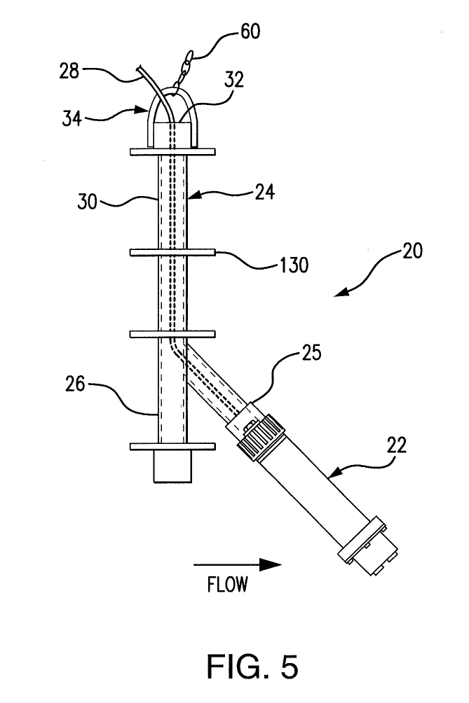

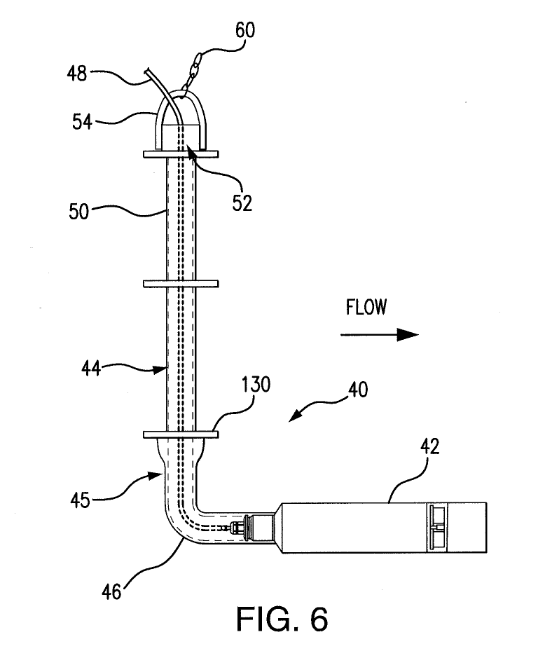

The present invention generally relates to a mount 100 or housing which may be attached to a mount for submersible analyzers or device that stabilize and protect such analyzers or devices, particularly when the analyzers are deep in the fluid (long drop) and/or in a high velocity fluid. A high velocity for fluid flow may be about 1-5 ft/sec, for example. The housing is preferably ruggedized meaning it is formed of a rigid support material that is also corrosion and weathering resistant and waterproof, and supports the analyzer or sensor to prevent breakage or damage thereof or prevent the analyzer or sensor rom being destroyed by the fluid in which it is submerged. The analyzers are configured to measure fluid contents at varying depths below a fixed structure 10, such as a platform or working deck. The uses of the mounting 100 of the present invention may include, but are not limited to, water or wastewater in treatment plants seeking to measure the levels of constituents well below the working surface or in fluid flows with high velocities at power plants, natural waterways, chemical plants or other industrial environments. The stability provided by mount 100 creates a stable platform leading to more accurate readings from the analyzers and devices while operating under conditions ranging from quiescent to turbulent. Mount 100 may be used to support any type of fluid analyzer or device, such as an ammonia analyzer 20 (FIG. 5) or a nitrate analyzer 40 (FIG. 6), for example. Mount 100 is also designed to allow for easy removal of the analyzer or device from the fluid, thereby facilitating the frequent maintenance often required for these types of analyzers.

The mount 100 according to the present invention provides additional functionality by providing part or all of the associated computation and/or connectivity including but not limited to chipset or industrial personal computer; and/or connectivity including and not limited to Wi-Fi, wireless, antenna, infrared, near field communication (NFC), Bluetooth, and/or communication protocols including and not limited to infrastructure protocols (ex: 6LowPAN, IPv4/IPv6, RPL), identification protocols (ex: EPC, uCode, IPv6, URIs), communications/transport protocols (ex: Wi-Fi, Bluetooth, LPWAN), discovery protocols (ex: Physical Web, mDNS, DNS-SD), data protocols (ex: MQTT, CoAP, AMQP, Websocket, Node), device management protocols (ex: TR-069, OMA-DM), semantic protocols (ex: JSON-LD, Web Thing Model), multi-layer framework protocol (ex: Alljoyn, IoTivity, Weave, Homekit), security protocol, process automation protocols (ex. AS-I, BSAP, CC-Link Industrial Networks, CIP (Common Industrial Protocol) common to DeviceNet, CompoNet, ControlNet and EtherNet/IP, ControlNet, DC-BUS, DeviceNet, DF-1, ControlLogix, CompactLogix, PLC-5, SLC-500, and MicroLogix class devices, DirectNet, EtherCAT, Ethernet Global Data (EGD), EtherNet/IP, Ethernet Powerlink, FINS, FOUNDATION fieldbus—H1 & HSE, HART Protocol, HostLink Protocol, Interbus, MECHATROLINK, MelsecNet, and MelsecNet II, /B, and/H, Modbus PEMEX, Modbus Plus, Modbus RTU or ASCII or TCP, OSGP—The Open Smart Grid Protocol, OpenADR, Optomux—Serial (RS-422/485), PieP, Profibus, PROFINET IO, RAPIEnet—Real-time Automation Protocols for Industrial Ethernet, Honeywell SDS, SERCOS III, Ethernet-based version of SERCOS real-time interface standard, SERCOS interface, Open Protocol for hard real-time control of motion and I/O, GE SRTP—GE Fanuc PLCs, Sinec H1—Siemens, SynqNet Danaher, TTEthernet—TTTech, MPI—Multi Point Interface) including but not limited to Bluetooth, BLE (Bluetooth Low Energy), ZigBee, Z-Wave, 6LoWPAN, Thread, WiFi, WiFi-ah (HaLow), 2G, 3G, 4G and 5G, LTE Cat 0, 1, & 3, LTE-M1, With LTE, NB-IoT, NFC, RFID, SigFox, LoRaWAN, Ingenu, Weightless-N, Weightless-P, Weightless-W, ANT & ANT+, DigiMesh, MiWi, EnOcean, Dash7, WirelessHART, and/or protocol translators and/or protocol convertors, and/or energy generation including and not limited to mini turbines, solar, water (hydrokinetic and potential), wind, or conversion of chemical to electrical energy; and/or storage including and not limited to battery or capacitance, that facilitates the hosting of a branded or generic sensor, analyzer or device, and the like.

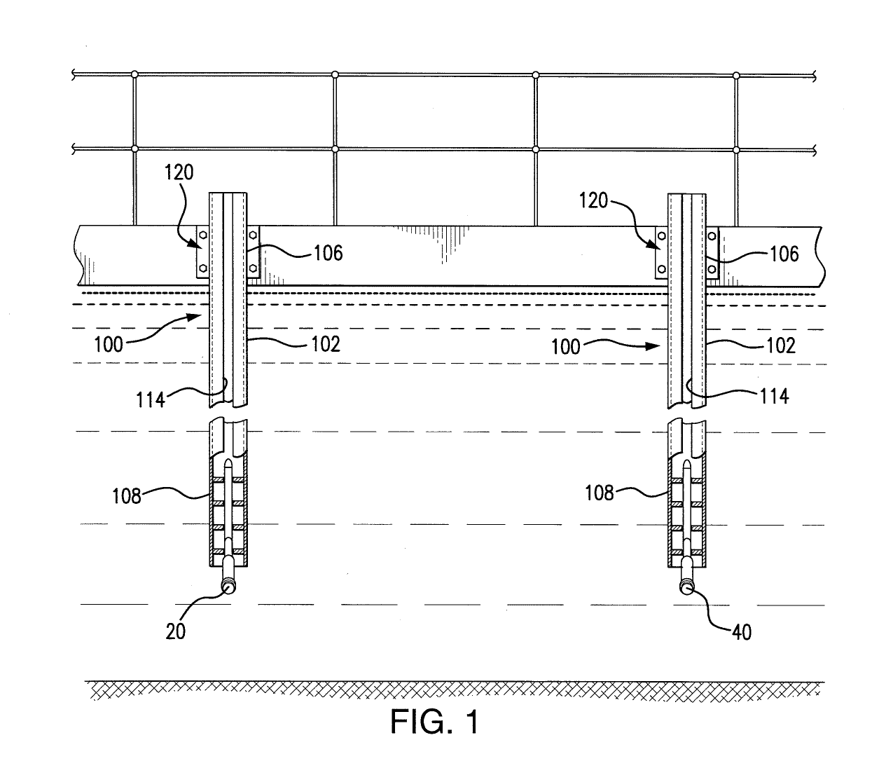

As seen in FIG. 1, mount 100 generally includes an elongated housing 102 that is designed to be submersed in the fluid and fixedly attached to a fixed surface, such as a platform or working deck. The housing 102 is elongated because its length is several times greater than its width or diameter. Mount 100 comprises a cylindrical body 104 with an upper end 106 configured to be mounted to the fixed surface 10 and an opposite lower end 108. Cylindrical body 104 is preferably ruggedized by being formed of a rigid metal, such as stainless steel. It will be appreciated that cylindrical body 104 may be formed of any material, whether metal or non-metallic, that has sufficient rigidity to maintain the stability of the analyzer or device submerged in the fluid. In a preferred embodiment, cylindrical body 104 is also formed of a substantially non-corrosive material.

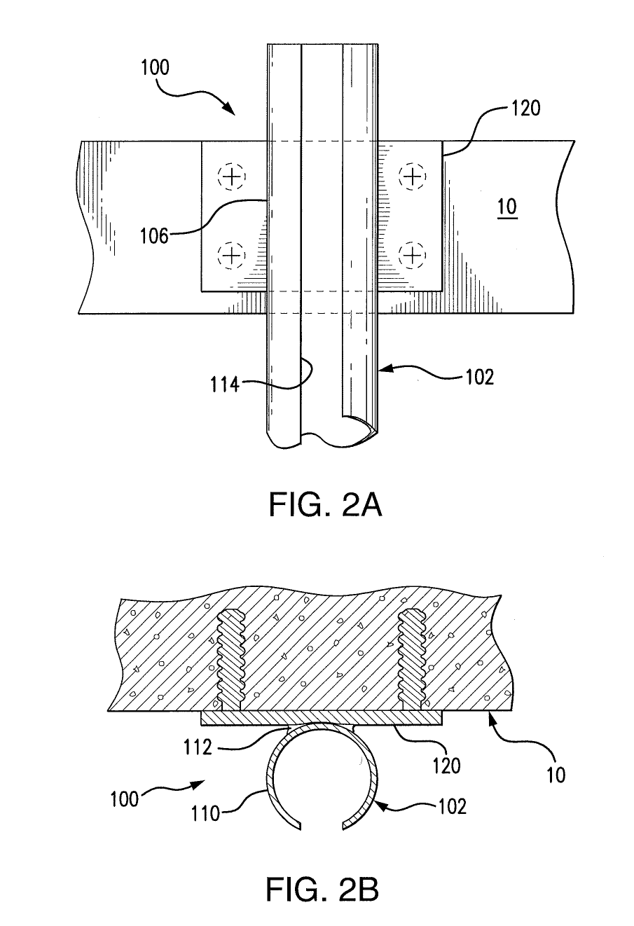

An attachment 112 fixedly attaches cylindrical body 104 to the fixed surface. Attachment 112 may be, for example, a bonding engagement, such as a weld between an outer surface 110 of cylindrical body 104 and base plate 120 on the fixed surface 10, as seen in FIGS. 2A and 2B. Base plate 120, may be, for example, a metal bracket mounted on the fixed surface via thread rods 122. It will be appreciated that attachment 112 may be any known attachment, as long as the attachment fixedly mounts housing 102 to the fixed surface 10

Housing 102 includes a slot 114 extending between upper and lower ends 106 and 108 along a longitudinal axis 116 of housing 102, as seen in FIGS. 2A and 2B. Slot 114 preferably opens or faces away from fixed surface 10 and toward the downstream flow direction to allow for proper orientation of the analyzer or device in the fluid when received in housing 102.

FIG. 1 shows mount 100 supporting a submersible ammonia analyzer 20 and a submersible nitrate analyzer 40, respectively. As seen in FIG. 5, the ammonia analyzer 20 includes an ammonia sensor 22 that is supported by an extension 24 at or near its bottom 26. Extension 24 may include an adapter 25 to facilitate coupling of the sensor 22 onto extension 24. Extension 24 may be a pipe, for example, through which the sensor's cabling 28 may be threaded through and out of an opening at its top 30 so that the cabling 28 may be connected to a terminal (not shown). The top 30 may also include a cap 32 that has a handle 34 for pulling the analyzer 20 out of housing 102. The cap 32 may have openings to allowing cabling 28 to pass therethough. The bottom 26 of extension 24 may also include a cap 36 that may have one or more drain holes. As seen in FIG. 5, the ammonia analyzer 20 preferably oriented downwardly at about 45 degrees to the flow direction

As seen in FIG. 6, the nitrate analyzer 40 includes a nitrate sensor 42 that is supported by an extension 44 at its bottom 46. Extension 44 may include an adapter 45 to facilitate coupling of the sensor 42 onto extension 44. Like extension 24 of the ammonia analyzer 20, extension 44 may be a pipe through which the sensor's cabling 48 may be threaded through and out of an opening at its top 50 so that the cabling 48 may be connected to a terminal (not shown). The top 50 may also include a cap 52 that has a handle 54 for pulling the analyzer 40 out of housing 102. The cap 52 may have openings to allowing cabling 48 to pass therethrough. Holes may be provided in the bottom 46 of the adapter 45 for drainage. As seen in FIG. 6, the nitrate analyzer 40 preferably oriented at about a 90 degree angle in the toward the flow direction.

Slot 114 of housing 102 is sized to receive a portion of extension 24 or 44, such as the bent or angled portion of the extension 24 or 44, as seen in FIGS. 5 and 6, respectively, which is supporting the sensor 22 or 44. Thus the width W (FIG. 2B) of slot 114 allows the analyzer 20 or 40 to be easily inserted or removed from housing 102 while the sensor 22 or 24 remains outside of the housing 102 because that portion of extension 24 or 44 extends through slot 114.

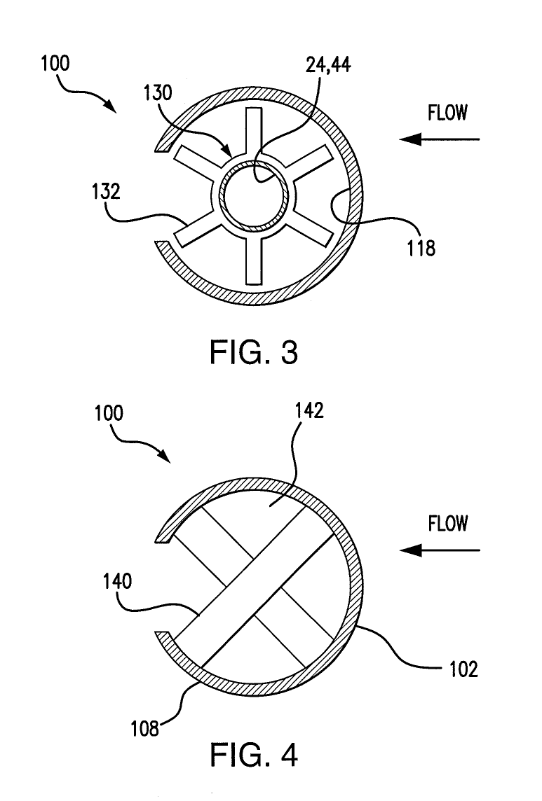

One or more spacers 130 are preferably provided on extension 24 or 44, as best seen in FIGS. 5 and 6, respectively. The spacers 130 surround the extension 24 or 44 and are space from one another along the longitudinal length of extension 24 or 44. Each spacer 130 may have one or more radial arms 132 (FIG. 3) such that the arms 132 extend between the extension 24 or 44 and an inner surface 118 of housing 102. In a preferred embodiment, at least three spacers 130 are provided on extension 24 or 44. Spacers 130 provide protection to the extensions 24 and 24 when inserting and retrieving the analyzer 20 or 40 or device from housing 102. Spacers 130 are preferably made of a non-metallic material.

The mount or submersible housing of the present invention provides additional functionality by providing part or all of the associated computation utilizing a computing device including and not limited to chipset or industrial personal computer; and/or a relay or communication device with wired or wireless connectivity to a computing device including but not limited to Wi-Fi, Bluetooth, antenna, infrared, or near field communication (NFC), wherein the computing device is capable of implementing complex protocols including but not limited to infrastructure protocols (ex: 6LowPAN, IPv4/IPv6, RPL), identification protocols (ex: EPC, uCode, IPv6, URIs), transport protocols (ex: Wi-Fi, Bluetooth, LPWAN), discovery protocols (ex: Physical Web, mDNS, DNS-SD), data protocols (ex: MQTT, CoAP, AMQP, Websocket, Node), device management protocols (ex: TR-069, OMA-DM), security protocols, semantic protocols (ex: JSON-LD, Web Thing Model), multi-layer framework protocol (ex: Alljoyn, IoTivity, Weave, Homekit), process automation protocols (ex. AS-I, BSAP, CC-Link Industrial Networks, CIP (Common Industrial Protocol) common to DeviceNet, CompoNet, ControlNet and EtherNet/IP, ControlNet, DC-BUS, DeviceNet, DF-1, ControlLogix, CompactLogix, PLC-5, SLC-500, and MicroLogix class devices, DirectNet, EtherCAT, Ethernet Global Data (EGD), EtherNet/IP, Ethernet Powerlink, FINS, FOUNDATION fieldbus—H1 & HSE, HART Protocol, HostLink Protocol, Interbus, MECHATROLINK, MelsecNet, and MelsecNet II, /B, and/H, Modbus PEMEX, Modbus Plus, Modbus RTU or ASCII or TCP, OSGP—The Open Smart Grid Protocol, OpenADR, Optomux—Serial (RS-422/485), PieP, Profibus, PROFINET IO, RAPIEnet—Real-time Automation Protocols for Industrial Ethernet, Honeywell SDS, SERCOS III, Ethernet-based version of SERCOS real-time interface standard, SERCOS interface, Open Protocol for hard real-time control of motion and I/O, GE SRTP—GE Fanuc PLCs, Sinec H1—Siemens, SynqNet—Danaher, TTEthernet—TTTech, MPI—Multi Point Interface) including and not limited to Bluetooth, BLE (Bluetooth Low Energy), ZigBee, Z-Wave, 6LoWPAN, Thread, WiFi, WiFi-ah (HaLow), 2G, 3G, 4G and 5G, LTE Cat 0, 1, & 3, LTE-M1, With LTE, NB-IoT, NFC, RFID, SigFox, LoRaWAN, Ingenu, Weightless-N, Weightless-P, Weightless-W, ANT & ANT+, DigiMesh, MiWi, EnOcean, Dash7, WirelessHART, and/or protocol translators and/or protocol convertors. In some embodiments the disclosure may be fitted with energy generation devices including and not limited to mini turbines, solar, water (hydrokinetic and potential), wind, or conversion of chemical to electrical energy. In other embodiments the disclosure may include energy storage devices including and not limited to battery or capacitance, which facilitates the hosting of a branded or generic sensor, device, or analyzer.

In some embodiments the mount or submersible housing can support the ruggedization of sensors, sensor mounts or any multifunctional part, including those used for computation (such as chips).

A method of stabilizing a submersible analyzer or device preferably includes the steps of fixedly mounting the upper end 106 of the elongated submersible housing 102 to the fixed structure 10 in a manner described above; coupling the one or more spacers 130 onto the extension 24 or 44 of the analyzer 20 or 40 or device; installing the analyzer 20 or 40 or device into the opening at the upper end 106 of the housing 102 so that a lower portion of the extension 24 or 44 passes through the slot 114 of the housing 102; and advancing the analyzer 20 or 40 or device down toward the lower end of the housing such that the sensor 22 or 42 is exposed outside of the housing near or at the lower end thereof and the one or more spacers are located between the extension 24 or 44 and the inner surface of the housing 102. The lower end 108 is preferably substantially closed, such as by cross-plates 140 (FIG. 4) to provide a stop for the analyzer when advanced downwardly in the housing 102. It will be appreciated that the lower end 108 may be at least partially closed in any manner as long as some openings, such as openings 142, remain to allow fluid to pass therethrough. The analyzer 20 or 40 or device may then be easily retrieved from the housing 102 by pulling the handle end 34 or 54, such as by a chain 60, of the extension 24 or 44 allowing the analyzer 20 or 40 or device to be removed from the housing 103 through the opening in the upper end 106 as the extension 24 or 44 and the analyzer 20 or 40 moves up the slot 114. The easy retrieval of the analyzer 20 or 40 facilitates the frequent maintenance typically required for these analyzers.

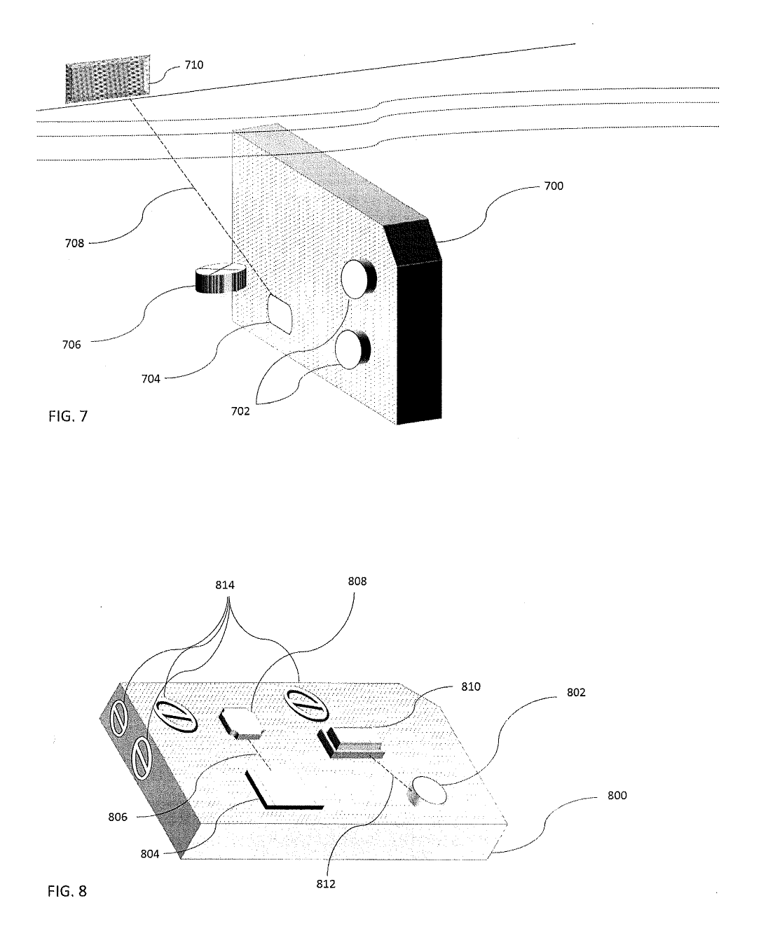

FIG. 7 shows a submersible housing 700 according to the present invention that shields one or more sensors 702. The sensors 702 may be velocity sensors. The sensors send data from the housing 700 utilizing a relay 704 and energy collected from an energy generation device (e.g. micro hydraulic turbines) 706, to wirelessly communicate 708 with a remote computer 710 located somewhere above the fluid being analyzed (for example, wastewater).

FIG. 8 illustrates a submersible housing 800 that shields one or more sensors 802. The housing 800 utilizes an energy generation device (e.g. solar panels) 804, to generate energy collected via wired means 806 in an energy storage device (e.g. battery) 808, in order to power an on-board computing device 810, which collects data direct from the sensor 802 utilizing wired means 812. The housing 800 further comprises multiple port-slots 814 for the insertion and removal of additional computing devices, energy generation devices, or storage devices, in order to facilitate the ruggedization of those devices.

A method of analyzing fluid according to the present invention from the housing 102, 700 or 800 of the present invention, comprising the steps of installing one or more sensors within the housing; installing one or more computing devices or relays to transmit a signal from the sensors to one or more remote computing devices; and placing the housing within the fluid such that the sensors transmit data regarding properties of the fluid to the computing devices. The method may also include the step of stabilizing the housing from the fixed structure 10 by fixedly mounting the upper end of the housing to the fixed structure 10; coupling one or more of the spacers 130 with the extension that supports the analyzer 20, 40, 702, or 802; installing the analyzer 20, 40, 702, or 802 or device 706, 804, or 808 into the opening at the upper end of the housing so that the lower portion of the extension passes through the slot, such as slot 114, extending between the upper end and the lower end of the housing along its longitudinal axis thereof; and advancing the analyzer down toward the lower end of the housing such that the one or more sensors are exposed outside of the housing near or at the lower end thereof and the at least one spacer is located between the extension and the inner surface of the housing.

In certain embodiments, the port slots on the submersible housing may be connected to port slots on a mount linking the two together and attaching the housing to said mount for stabilization purposes. In some such embodiments, the slots on the mount and/or submersible housing may include a means to connect power or data from or to the submersible housing, including but not limited to a quick connect, plug or outlet.

In certain embodiments, the submersible housing may include a display panel which can be used to display information regarding the housed sensors, devices or analyzers. Data related to these sensors, devices or analyzers may be cycled using one or more buttons on the housing. In some such embodiments the display panel may be an interface panel which can be used to control any of the sensors, analyzers or devices for purposes including but not limited to a sensor taking an immediate reading, changing the type of information being sensed, taking extended readings, a device latching into place, locking into position, unlocking, sending collected energy or powering on, or an analyzer analyzing collected data, preparing to analyze data to be collected at a prescribed time, or changing the type of data being analyzed.

The present invention is applicable to the design, installation, and use of multifunctional stabilizing mounts, or attachments to mounts, and for in special cases a submersible or semi-submersible analyzers (sensors and probes and their inputs and outputs), broadly in any fluid environment (gas, such as air, or liquid, such as water), and particularly in environments where the anticipated velocity of fluids is substantial and/or the mounting depth is of the analyzers is significant. Such analyzers may be but are not limited to sensors or probes for measuring, for example, ammonia, nitrates or nitrites in environments, such as and not limited to water, wastewater, rivers, lakes, reservoirs, distribution and collection systems, chemical plants, or power plants, hospitals and hotels; and in air (or example, nitrogen oxides, sulfur oxides and particulates) for cities, towns, industries, factories and in the indoor air environment. The sensing can be visual or spectral (such as a camera or any spectral imaging such as ultra-violet, infra-red, Raman or FTIR), auditory (sound), vibratory, potentiometric, or in the chemical environment (analysis of chemical species).

While particular embodiments have been chosen to illustrate the invention, it will be understood by those skilled in the art that various changes and modifications can be made therein without departing from the scope of the invention as defined in the appended claims.

What is claimed is:

1. A mount for supporting a submersible or semi-submersible analyzer or device, comprising:
an elongated submersible or semi-submersible housing for supporting an analyzer or device, the housing having a geometric body with upper and lower ends, the upper end being configured to mount to a fixed structure, and the upper end being open to receive the analyzer; and
a slot extending between the upper and lower ends of the elongated submersible or semi-submersible housing along a longitudinal axis of the elongated submersible or semi-submersible housing, the slot being sized to accept a portion of a sliding extension that supports a sensor of the analyzer,
wherein the elongated submersible or semi-submersible housing is ruggedized.

2. A mount according to claim 1, further comprising
an attachment that fixedly mounts the housing to the fixed structure.

3. A mount according to claim 2, wherein
the attachment is a bonded engagement between an outer surface of the housing and a base plate on the fixed structure.

4. A mount according to claim 1, wherein
the upper end of the housing has an opening for installing the analyzer or device and the lower end is at least partially closed.

5. A mount according to claim 1, further comprising
at least one spacer disposed around the extension, the spacer having at least one radial arm that extends between the extension and the inner surface of the housing.

6. A mount according to claim 1, further comprising
at least three spacers disposed around the extension, the spacers being spaced from one another and each spacer having at least one radial arm that extends between the extension and the inner surface of the housing.

7. A mount according to claim 1, wherein
the upper end of the housing has an opening for installing the analyzer or device and the lower end is substantially closed.

8. A method of analyzing fluid from a submersible or semi-submersible housing, comprising the steps of:
installing one or more sensors within the housing;
installing one or more local computing devices or installing one or more relays or transmitters to transmit a signal from said one or more sensors to one or more remote computing devices or a cloud network,
said local and remote computing devices communicating with said one or more sensors either through wired means or wireless means; and
positioning the housing such that the one or more sensors is in fluid and transmits data regarding properties of the fluid to said one or more local or remote computing devices.

9. A method according to claim 8, further comprising the steps of:
stabilizing the housing from a fixed structure by:
fixedly mounting an upper end of the housing to the fixed structure, the housing having a geometric body that is ruggedized;
coupling at least one spacer with a sliding extension that supports a sensor of an analyzer or device;
installing the analyzer or device into an opening at the upper end of the housing so that a lower portion of the extension passes through a slot extending between the upper end and a lower end of the housing along a longitudinal axis thereof; and
advancing the analyzer or device down toward the lower end of the housing such that the sensor is exposed outside of the housing near or at the lower end thereof and the at least one spacer is located between the extension and an inner surface of the housing.

10. A method according to claim 8, further comprising the step of
utilizing the one or more computing devices to implement complex protocols.

11. A method according to claim 8, further comprising the steps of
coupling an energy generation device to the housing for the generation of energy; and
installing an energy storage device for the collection of energy.

12. A method according to claim 8, further comprising the step of
sending a signal from the housing to the one or more remote computing devices, wherein the wireless means of communication is near field communication or an antenna.

13. A method according to claim 8, further comprising the step of
providing one or more rigid port-slots in the housing for insertion and removal of one or more computing devices, energy storage devices or energy generation devices, wherein the one or more port-slots are integrated into the housing in order to facilitate the ruggedization of the sensors and the devices;
said port slots connecting the submersible housing to the mount incorporates circuitry to facilitate the transmission of power or data from or to the submersible housing.

14. A submersible or semi-submersible housing, comprising:
a ruggedized housing body supporting,
one or more sensors,
one or more relays capable of communicating with one or more computing devices,
one or more port-slots for connecting the ruggedized housing body to a mount or one or more slots for the installation and removal of the one or more sensors wherein the slots are rigidly integrated into the housing to facilitate the ruggedization of the one or more sensors, and
one or more computing devices capable of implementing complex protocols, or any number of energy storage devices, or any number of energy generation devices.

15. A submersible or semi-submersible housing according to claim 14, further comprising
an interface panel for direct control of any of the sensors therein, and
any number of buttons for cycling between the various sensors, display options for the various sensors, or the sensors and display options of the sensors,

16. A submersible or semi-submersible housing according to claim 14, wherein
said one or more sensors are velocity sensors; or
said any number of energy generation devices are micro hydraulic turbines.

17. A submersible or semi-submersible housing according to claim 15, wherein
said one or more computing devices are supported by said housing body,
wherein the one or more sensors sends a signal to said one or more computing devices directly through either wired or wireless means.

18. A submersible or semi-submersible housing according to claim 14, wherein
said housing body includes at least one of said any number of an energy generation device.

19. A submersible or semi-submersible housing according to claim 14, wherein
the housing body includes at least one of said any number of energy storage device.

20. A submersible or semi-submersible housing according to claim 14, wherein
said housing body includes said one or more slots for insertion and removal of a plurality of computing devices, energy storage devices, or energy generation devices, wherein the one or more port-slots are integrated into the housing in order to facilitate the ruggedization of said one or more sensors.
